(19) United States
(12) Patent Application Publication
(10) Pub. No.: US 2025/0387076 A1
(43) Pub. Date: Dec. 25, 2025

(54) METHOD AND SYSTEM FOR DETERMINING SPINAL CURVATURE

(71) Applicant: MOMENTUM HEALTH INC., Montreal (CA)

(72) Inventors: Evan DIMENTBERG; Jean OUELLET, Montreal (CA); Philippe MILLER, Montreal (CA); Frank DE WIJK, Amsterdam (NL); Leander GOOR, Groningen (NL)

(73) Assignee: MOMENTUM HEALTH INC., Montreal, QC (CA)

(21) Appl. No.: 18/880,483

(22) PCT Filed: Aug. 11, 2023
(86) PCT No.: PCT/IB2023/058155

§ 371 Date: Dec. 31, 2024

Related U.S. Application Data
(60) Provisional application No. 63/371,081, filed on Aug. 11, 2022.

(51) Int. Cl. A61B 5/00; A61B 5/107; G06T 7/00; G06T 7/11; G06T 7/55; G06T 7/73; G06T 17/00

(52) U.S. Cl. CPC A61B 5/4561; A61B 5/1071; A61B 5/1073; A61B 5/1079; A61B 5/7264; G06T 7/0014; G06T 7/11; G06T 7/55; G06T 7/73; G06T 17/00; G06T 2207/10016; G06T 2207/20084; G06T 2207/30012; G06T 2207/30204; G06T 2210/41

(57) ABSTRACT

A method and a system for determining a degree of spinal curvature includes, for each segment of a plurality of segments of a torso of a subject: determining at least one volume of the segment; and determining a degree of spinal curvature of the subject based on the volumes of the respective segments. Another method of determining a degree of spinal curvature includes: receiving a plurality of images of at least a torso of a subject; determining a plurality of segments of the torso; determining respective volumes of the plurality of segments of the torso based on the plurality of images; and determining a degree of spinal curvature of the subject based on the respective shares of the volumes of the segments; and outputting the determined degree of spinal curvature.

### CROSS-REFERENCE TO RELATED APPLICATIONS

This application claims priority on U.S. Provisional Application No. 63/371,081 filed on Aug. 11, 2022, entitled “METHOD OF DETERMINING SPINAL CURVATURE”, the content of which is hereby incorporated by reference in their entirety.

### TECHNICAL FIELD

The present technology relates to determining the shape of a human body, and in particular to determining human spinal curvature.

### BACKGROUND

Scoliosis is a condition in which a subject has an abnormal spinal curvature. For the purpose of diagnosis and treatment, it is important to be able to quantify the subject's spinal curvature and its changes over time. One measure of the deviation from normal spinal curvature is known as the Cobb angle.

Determining the Cobb angle typically requires the analysis of X-ray images of the subject. As a result, this determination requires the subject to get new X-rays whenever a new or updated measurement is desired. The subject must attend at a hospital or other facility with X-ray imaging equipment, and be exposed to the radiation inherent in the X-ray imaging process. In addition, this process is time-consuming for both the subject and the radiologist who must analyze the images.

There is a need for a method of determining the spinal curvature of a scoliosis subject, in particular the Cobb angle, that is simpler, less time-consuming, and/or safer for the subject.

### SUMMARY

It is an object of the present technology to mitigate one or more disadvantages of the prior art.

It is an object of the present technology to provide a method of determining a Cobb angle of a subject without using X-ray imaging.

It is an object of the present technology to provide a method of determining a Cobb angle of a subject that does not require specialized imaging equipment.

It is an object of the present technology to provide a method of determining a Cobb angle of a subject that is easier and less time-consuming.

According to a first broad aspect, there is provided a method of determining a degree of spinal curvature, the method being executed by a processor, the method comprising: for each segment of a plurality of segments of a torso of a subject: determining at least one volume of the segment; and determining the degree of spinal curvature of the subject based on the volumes of the respective segments.

In one embodiment, the at least one volume is a volume of a substantially planar segment oriented in a transverse plane of the subject.

In one embodiment, the at least one volume includes four volumes of the segment in respective quadrants of the transverse plane.

In one embodiment, the respective quadrants are defined relative to a center of a segment located inferior to a spine of the subject.

In one embodiment, the step of determining the degree of spinal curvature comprises determining a Cobb angle.

In one embodiment, the step of determining the degree of spinal curvature includes using a convolutional neural network.

In one embodiment, the convolutional neural network includes two convolutional layers, a flatten layer, and two dense layers, using an Adam optimizer.

In one embodiment, the method further comprises determining the plurality of segments, wherein said determining the plurality of segments comprises: obtaining a plurality of images of the torso of the subject; determining a shape of the torso based on the plurality of images; and dividing the shape of the torso into the plurality of segments.

In one embodiment, at least some of the plurality of images contain at least one reference marker of a plurality of reference markers, and determining the shape of the torso includes determining a position of at least one point on the torso with respect to the plurality of reference markers.

In one embodiment, the step of determining the shape of the torso comprises generating a three-dimensional (3D) model of the torso.

According to a second broad aspect, there is provided a method of determining a degree of spinal curvature, the method being executed by a processor, the method comprising: receiving a plurality of images of at least a torso of a subject; determining a plurality of segments of the torso; determining respective volumes of the plurality of segments of the torso based on the plurality of images; determining the degree of spinal curvature of the subject based on respective shares of the respective volumes of the segments; and outputting the determined degree of spinal curvature.

In one embodiment, the step of determining the degree of spinal curvature comprises using a convolutional neural network.

In one embodiment, the convolutional neural network includes two convolutional layers, a flatten layer, and two dense layers, using an Adam optimizer.

In one embodiment, the step of determining the plurality of segments comprises: determining a shape of the torso based on the plurality of images; and dividing the shape of the torso into the plurality of segments.

In one embodiment, at least some of the plurality of images include at least one of a plurality of reference markers, and wherein determining the shape of the torso includes determining a position of at least one point on the torso with respect to the plurality of reference markers.

In one embodiment, the step of determining the position of the at least one point on the torso includes generating a depth map of a plurality of points on the torso.

In one embodiment, the step of determining the shape of the torso comprises generating a three-dimensional (3D) model of the torso.

In one embodiment, the plurality of segments are substantially planar segments each oriented in a transverse plane of the subject.

In one embodiment, each one of the plurality of segments is located in one quadrant of the transverse plane.

In one embodiment, the quadrant is defined relative to a center of a segment located inferior to a spine of the subject.

In one embodiment, the plurality of images include at least one video recording.

According to a further broad aspect, there is provided a system for determining a degree of spinal curvature, the system comprising: a processor; a non-transitory storage medium operatively connected to the processor, the non-transitory storage medium comprising computer-readable instructions; the processor, upon executing the instructions, being configured to: for each segment of a plurality of segments of a torso of a subject: determine at least one volume of the segment; and determine the degree of spinal curvature of the subject based on the volumes of the respective segments.

In one embodiment, the at least one volume is a volume of a substantially planar segment oriented in a transverse plane of the subject.

In one embodiment, the at least one volume includes four volumes of the segment in respective quadrants of the transverse plane.

In one embodiment, the respective quadrants are defined relative to a center of a segment located inferior to a spine of the subject.

In one embodiment, the processor is further configured to determine the degree of spinal curvature by determining a Cobb angle.

In one embodiment, the processor is further configured to determine the degree of spinal curvature by using a convolutional neural network.

In one embodiment, the convolutional neural network includes two convolutional layers, a flatten layer, and two dense layers, using an Adam optimizer.

In one embodiment, the processor is further configured to determine the plurality of segments by: obtaining a plurality of images of the torso of the subject; determining a shape of the torso based on the plurality of images; and dividing the shape of the torso into the plurality of segments.

In one embodiment, at least some of the plurality of images contain at least one reference marker of a plurality of reference markers, and determining the shape of the torso includes determining a position of at least one point on the torso with respect to the plurality of reference markers.

In one embodiment, the processor is further configured to determine the shape of the torso by generating a three-dimensional (3D) model of the torso.

According to still another broad aspect, there is provided a system for determining a degree of spinal curvature, the system comprising: a processor; a non-transitory storage medium operatively connected to the processor, the non-transitory storage medium comprising computer-readable instructions; the processor, upon executing the instructions, being configured to: receive a plurality of images of at least a torso of a subject; determine a plurality of segments of the torso; determine respective volumes of the plurality of segments of the torso based on the plurality of images; determine the degree of spinal curvature of the subject based on respective shares of the volumes of the segments; and output the determined degree of the spinal curvature.

In one embodiment, the processor is configured to determine the degree of spinal curvature using a convolutional neural network.

In one embodiment, the convolutional neural network includes two convolutional layers, a flatten layer, and two dense layers, using an Adam optimizer.

In one embodiment, the processor is configured to determine the plurality of segments by: determining a shape of the torso based on the plurality of images; and dividing the shape of the torso into the plurality of segments.

In one embodiment, at least some of the plurality of images include at least one of a plurality of reference markers, and wherein the processor is configured to determine the shape of the torso by determining a position of at least one point on the torso with respect to the plurality of reference markers.

In one embodiment, the processor is further configured to determine the position of the at least one point on the torso by generating a depth map of a plurality of points on the torso.

In one embodiment, the processor is further configured to determine the shape of the torso by generating a three-dimensional (3D) model of the torso.

In one embodiment, the plurality of segments are substantially planar segments each oriented in a transverse plane of the subject.

In one embodiment, each of the plurality of segments is located in one quadrant of the transverse plane.

In one embodiment, the quadrant is defined relative to a center of a segment located inferior to a spine of the subject.

In one embodiment, the plurality of images include at least one video recording.

In the context of the present specification, a “server” is a computer program that is running on appropriate hardware and is capable of receiving requests (e.g., from computing devices) over a network (e.g., a communication network), and carrying out those requests, or causing those requests to be carried out. The hardware may be one physical computer or one physical computer system, but neither is required to be the case with respect to the present technology. In the present context, the use of the expression a “server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “computing device” is any computing apparatus or computer hardware that is capable of running software appropriate to the relevant task at hand. Thus, some (non-limiting) examples of computing devices include general purpose personal computers (desktops, laptops, netbooks, etc.), mobile computing devices, smartphones, and tablets, and network equipment such as routers, switches, and gateways. It should be noted that a computing device in the present context is not precluded from acting as a server to other computing devices. The use of the expression “a computing device” does not preclude multiple computing devices being used in receiving/sending, carrying out or causing to be carried out any task or request, or the consequences of any task or request, or steps of any method described herein. In the context of the present specification, a “client device” refers to any of a range of end-user client computing devices, associated with a user, such as personal computers, tablets, smartphones, and the like.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “server” and “third server” is not intended to imply any particular order, type, chronology, hierarchy or ranking (for example) of/between the server, nor is their use (by itself) intended imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

Having thus generally described the nature of the technology, reference will now be made to the accompanying drawings, showing by way of illustration example implementations thereof and in which:

FIG. 1 depicts a schematic diagram of a computing device in accordance with one or more non-limiting implementations of the present technology;

FIG. 2 depicts a schematic diagram of a communication system in accordance with one or more non-limiting implementations of the present technology;

FIG. 3 is a flowchart of a method in accordance with one or more non-limiting implementations of the present technology;

FIG. 4 is an illustration of one method of taking images of a subject in accordance with one or more non-limiting implementations of the present technology;

FIG. 5 is a flowchart of a method of processing images of a subject in accordance with one or more non-limiting implementations of the present technology;

FIG. 6 illustrates a sparse cloud representing a three-dimensional (3D) model of the subject generated from tie points, in accordance with one or more non-limiting implementations of the present technology;

FIG. 7 illustrates a depth map of the points, in accordance with one or more non-limiting implementations of the present technology;

FIG. 8 is a flowchart of a method of calculating volumes in accordance with one or more non-limiting implementations of the present technology;

FIG. 9 illustrates the sliced 3D model, in accordance with one or more non-limiting implementations of the present technology; and

FIG. 10 is a flowchart of a method of determining a volume of a segment in accordance with one or more non-limiting implementations of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In one or more non-limiting implementations of the present technology, the processor may be a general purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

With these fundamentals in place, we will now consider some non-limiting examples to illustrate various implementations of aspects of the present technology.

### Computing Device

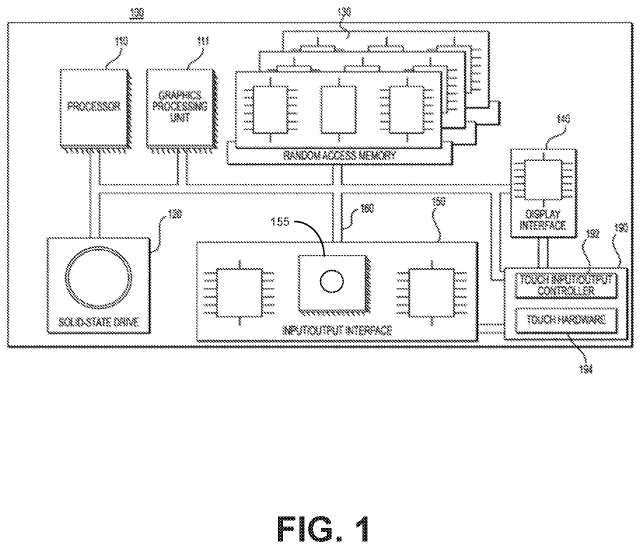

Referring to FIG. 1, there is shown a computing device 100 suitable for use with some implementations of the present technology, the computing device 100 comprising various hardware components including one or more single or multi-core processors collectively represented by processor 110, a graphics processing unit (GPU) 111, a solid-state drive 120, a random-access memory 130, a display interface 140, and an input/output interface 150.

Communication between the various components of the computing device 100 may be enabled by one or more internal and/or external buses 160 (e.g., a PCI bus, universal serial bus, IEEE 1394 “Firewire” bus, SCSI bus, Serial-ATA bus, etc.), to which the various hardware components are electronically coupled.

The input/output interface 150 may include an imaging device 155. The imaging device may be a 3D camera configured to generate high-quality depth information, enabling the creation of immersive and realistic three-dimensional representations. The imaging device 155 may include a set of advanced hardware components and software algorithms that work together to capture and process three-dimensional data. By way of example, the imaging device 155 may include one or more lenses, filters, and sensors specifically tailored for capturing depth-related information. Further, the imaging device 155 may incorporate a depth sensor responsible for capturing depth data from a scene. This depth sensor module may utilize various depth sensing technologies, such as structured light, time-of-flight (ToF), stereo vision, or other advanced techniques. Optionally, the imaging device 155 may include an image sensor to capture traditional 2D images. The image sensor may be a CMOS (Complementary Metal-Oxide-Semiconductor) or CCD (Charge-Coupled Device) sensor, capable of capturing high-resolution images.

The input/output interface 150 may be coupled to a touchscreen 190 and/or to the one or more internal and/or external buses 160. The touchscreen 190 may be part of the display. In one or more implementations, the touchscreen 190 is the display. The touchscreen 190 may equally be referred to as a screen 190. In the implementations illustrated in FIG. 1, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In one or more implementations, the input/output interface 150 may be connected to a keyboard (not shown), a mouse (not shown) or a trackpad (not shown) allowing the user to interact with the computing device 100 in addition or in replacement of the touchscreen 190.

According to implementations of the present technology, the solid-state drive 120 stores program instructions suitable for being loaded into the random-access memory 130 and executed by the processor 110 and/or the GPU 111 for determining a degree of spinal curvature. For example, the program instructions may be part of a library or an application.

The computing device 100 may be implemented as a server, a desktop computer, a laptop computer, a tablet, a smartphone, a personal digital assistant or any device that may be configured to implement the present technology, as it may be understood by a person skilled in the art.

### System

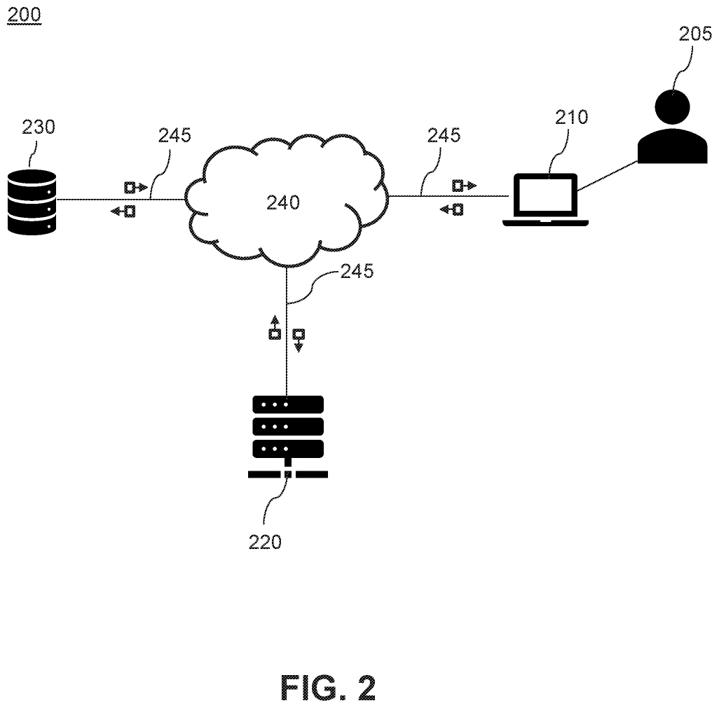

Referring to FIG. 2, there is shown a schematic diagram of a system 200, the system 200 being suitable for implementing one or more non-limiting implementations of the present technology. It is to be expressly understood that the system 200 as shown is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 200 may provide in certain instances simple implementations of the present technology, and that where such is the case they have been presented in this manner as an aid to understanding. As persons skilled in the art would understand, various implementations of the present technology may be of a greater complexity.

The system 200 comprises inter alia a client device 210 associated with a user 205, a server 220, and a database 230 communicatively coupled over a communications network 240.

### Client Device

The system 200 comprises a client device 210. The client device 210 is associated with the user 205. As such, the client device 210 can sometimes be referred to as a “computing device”, “end user device” or “client computing device”. It should be noted that the fact that the client device 210 is associated with the user 205 does not need to suggest or imply any mode of operation-such as a need to log in, a need to be registered, or the like.

The client device 210 comprises one or more components of the computing device 100 such as one or more single or multi-core processors collectively represented by processor 110, the graphics processing unit (GPU) 111, the solid-state drive 120, the random access memory 130, the display interface 140, and the input/output interface 150.

It will be appreciated that the client device 210 may be implemented as a server, a desktop computer, a laptop, a smartphone and the like.

In one or more implementations, the client device 210 is configured to execute a browser application. The purpose of the given browser application is to enable the user 205 to access one or more web resources. How the given browser application is implemented is not particularly limited. Non-limiting examples of the given browser application that is executable by the client device 210 include Google™ Chrome™, Mozilla™ Firefox™, Microsoft™ Edge™, and Apple™ Safari™.

### Server

It will be appreciated that the server 220 can be implemented as a conventional computer server and may comprise at least some of the features of the computing device 100 shown in FIG. 1. In a non-limiting example of one or more implementations of the present technology, the server 220 is implemented as a server running an operating system (OS). Needless to say that the server 220 may be implemented in any suitable hardware and/or software and/or firmware or a combination thereof. In the disclosed non-limiting implementation of present technology, the server 220 is a single server. In one or more alternative non-limiting implementations of the present technology, the functionality of the server 220 may be distributed and may be implemented via multiple servers (not shown).

The implementation of the server 220 is well known to the person skilled in the art. However, the server 220 comprises a communication interface (not shown) configured to communicate with various entities (such as the database 230, for example and other devices potentially coupled to the communication network 240) via the communication network 240. The server 220 further comprises at least one computer processor (e.g., the processing device of the computing device 100) operationally connected with the communication interface and structured and configured to execute various processes to be described herein.

### Database

A database 230 is communicatively coupled to the server 220 and the client device 210 via the communications network 240 but, in one or more alternative implementations, the database 230 may be directly coupled to the server 220 without departing from the teachings of the present technology. Although the database 230 is illustrated schematically herein as a single entity, it will be appreciated that the database 230 may be configured in a distributed manner, for example, the database 230 may have different components, each component being configured for a particular kind of retrieval therefrom or storage therein.

The database 230 may be a structured collection of data, irrespective of its particular structure or the computer hardware on which data is stored, implemented or otherwise rendered available for use. The database 230 may reside on the same hardware as a process that stores or makes use of the information stored in the database 230 or it may reside on separate hardware, such as on the server 220. The database 230 may receive data from the server 220 for storage thereof and may provide stored data to the server 220 for use thereof.

### Communication Network

In one or more implementations of the present technology, the communications network 240 is the Internet. In one or more alternative non-limiting implementations, the communication network 240 may be implemented as any suitable local area network (LAN), wide area network (WAN), a private communication network or the like. It will be appreciated that implementations for the communication network 240 are for illustration purposes only. How a communication link 245 (not separately numbered) between the client device 210, the server 220, the database 230, and/or another computing device (not shown) and the communications network 240 is implemented will depend inter alia on how each computing device is implemented.

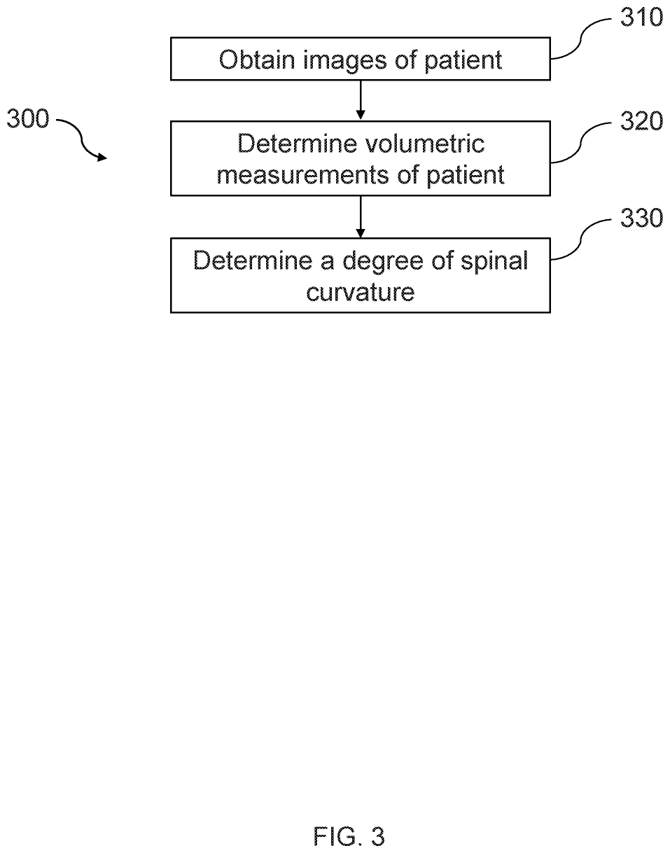

FIG. 3 depicts a flowchart of a method 300 of determining a spinal curvature of a subject, in accordance with one or more non-limiting implementations of the present technology.

In some implementations, the method 300 may be implemented by the client device 210. In other implementations, the method 300 may be implemented by the server 220. In yet another implementation, at least some of the steps associated with the method 300 may be implemented by the client device 210 while the remaining steps may be implemented by the server 220. It is to be noted that where the method 300 is to be implemented should not limit the scope of present disclosure.

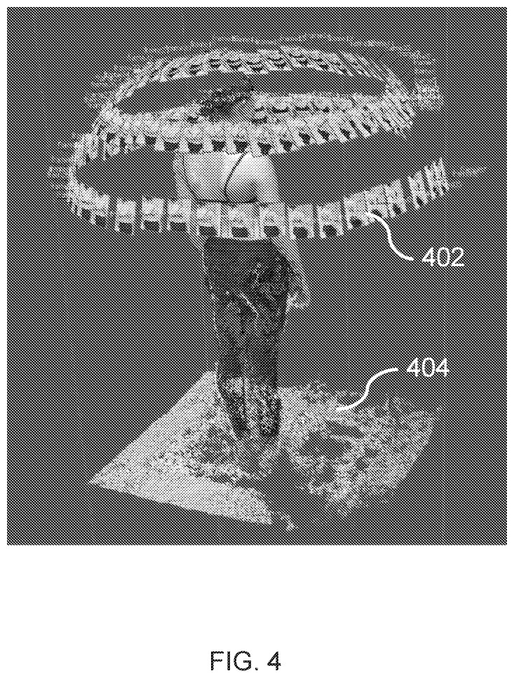

The method 300 commences at step 310, where based on an input from the user 205, the imaging device 155 may capture multiple images or pictures of the subject. The subject may be subject wearing no or minimal clothing on the upper body. The images may be images of at least the subject's torso taken around the torso, however it is contemplated that other parts of the subject's body may be imaged instead if the desired information relates to that part of the body. The images may be frames from a video taken using the imaging device 155 or any other suitable video recording equipment. The images may be taken from multiple angles, for example by the user 205 walking around the subject while recording a video using the imaging device 155, as illustrated schematically in FIG. 4 by a series of video frames 402. The video may have a high resolution and a high frame rate such as 30 or 60 frames per second.

In some implementations, the images may be captured in sets of images around the subject. In one example, one set of images may be captured around the subject in which the whole torso is visible. In another example, at least two sets of images may be captured, each set taken around the subject at a different height along the torso so as to image a respective portion of the torso. After capturing the two sets of images, the processor 110 and/or the GPU 111 may combine the two sets of images to obtain a single set of images in which the whole torso is visible. It is to be noted that how the images of torso are captured or obtained should not limit the scope of present disclosure.

In some implementations, markers are positioned at predefined locations around the subject before the images are taken so that the markers appear in the images of the subject. It should be understood that the relative positions between the markers is known.

In one implementation, the subject may stand on a sheet during the recording process. The sheet may be a square or rectangular sheet 404 such as a sheet of A0 or A1 paper, which may have a marker on each of the corners, the purpose of which will be described below. The markers may be distinct markers, such as 12-bit circular coded targets, or may be otherwise visually distinguishable from each other. Alternatively, markers may be placed on the body of the subject. It is contemplated that fewer markers, for example three markers, or more than four markers, may be used. The user 205 recording the images or video may attempt to capture one or more of the markers in as much of the recording as possible.

At step 320, the processor 110 and/or the GPU 111 may process the images captured by the imaging device 155 to obtain volumetric measurements of the subject. Volumetric measurement is a process of quantifying the three-dimensional space occupied by the subject's torso. The volumetric measurements may take into account length, width, and height to determine the total volume of the subject's torso in space.

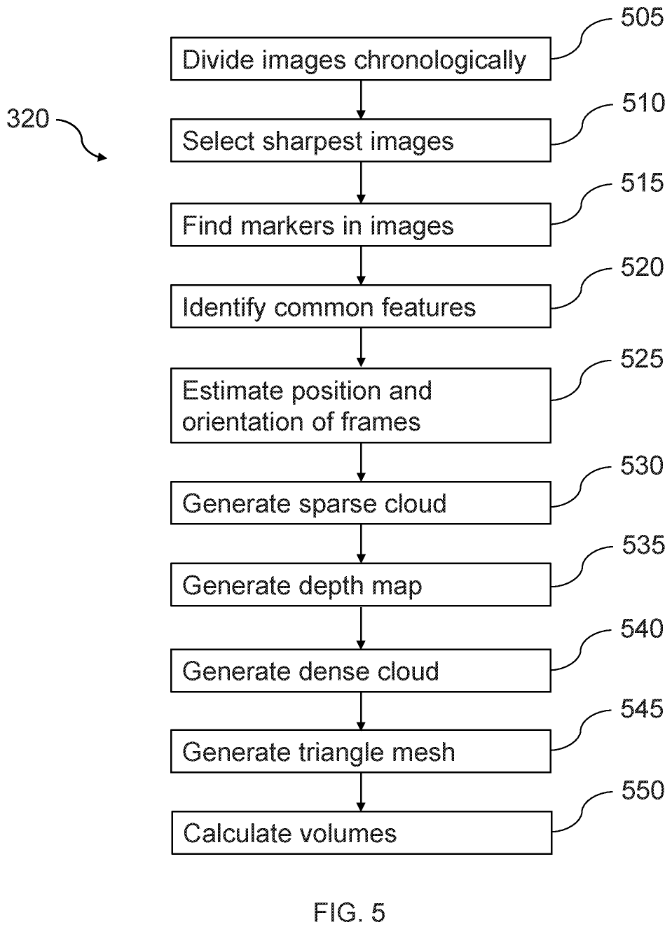

One exemplary method 320 of processing the images to obtain volumetric measurements is shown in more detail in FIG. 5. It should be understood that any suitable method of obtaining volumetric measurements of the subject may be used.

Referring to FIG. 5, the step of 320 obtaining the volumetric measurements, according to an implementation, begins at step 505 where the processor 110 and/or the GPU 111 may divide the images chronologically into groups or bins, for example 20 bins of equal size corresponding to 20 consecutive, equal time periods of the video. In this manner, mathematical or computing operations may be performed on each bin, such as ranking or selecting the most usable images, while ensuring that some images will be selected from each of the time periods. This may be useful, for example, if the video pans from the top to the bottom of the subject's torso, to ensure that information is retained for different portions of the torso.

At step 510, the processor 110 and/or the GPU 111 may select the sharpest images in each bin, for example the sharpest 20% of images in each bin. In some implementations, the processor 110 and/or the GPU 111 may compute a fast Fourier transform (FFT) of each frame, and convolve the image with a Laplacian kernel, and sort the images by the resulting quantity. It is contemplated that other suitable methods of determining image sharpness may be used, such as calculating the normalized summation of the squared pixel values, sometimes known as calculating the “energy” of the image. The images that are not selected may be discarded. Alternatively, images may be retained or discarded based on being sharper or less sharp than a sharpness threshold, for example a threshold value of the convolution of the FFT with the Laplacian kernel. Alternatively, the images may be selected in some other way, for example at regular intervals or at random intervals, instead of based on sharpness.

At step 515, the processor 110 and/or the GPU 111 may analyze the selected images to find the markers, for example the 12-bit coded targets on the sheet of paper previously described. The markers may be identified using a scale-invariant feature transform (SIFT) algorithm, for example. These markers serve as identifiable tie points that are common to multiple frames. The recognition of the markers may be done by a suitable commercial software such as Agisoft Metashape™.

At step 520, the processor 110 and/or the GPU 111 may compare the images to identify additional tie points, for example common features of the subject that are identifiable in more than one frame. In some implementations, the processor 110 and/or the GPU 111 may identify the tie points using a SIFT algorithm.

At step 525, using the identified tie points, the processor 110 and/or the GPU 111 may estimate the relative position and orientation of the imaging device 155 at the time of the frames.

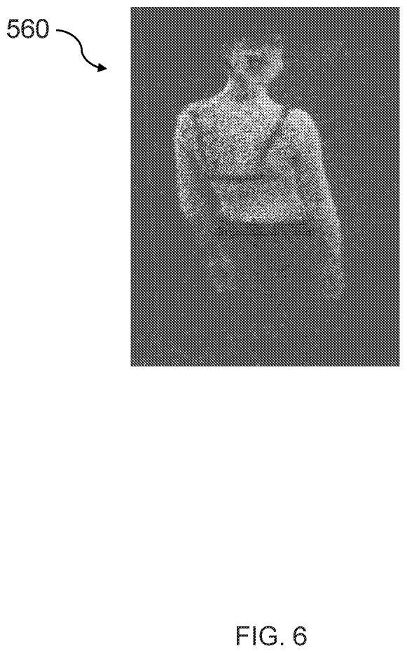

At step 530, the processor 110 and/or the GPU 111 may use the relative position and orientation of the imaging device 155 and generate a three-dimensional (3D) model of the subject's torso. In some implementations, the 3D model may be a sparse cloud of the points in space corresponding to the tie points. FIG. 6 illustrates the sparse cloud 560 representing a 3D model of the subject generated from tie points, in accordance with one or more non-limiting implementations of the present technology. In one example, the processor 110 and/or GPU 111 may rely on a suitable commercial software such as Agisoft Metashape™ to generate the sparse cloud 560. The orientation of the sparse cloud 560 may be determined based on the locations of the markers. For example, if the subject was facing in a designated direction, such as toward a specified edge of the sheet, or if an identifiable marker was placed on the front or back of the subject's body, two horizontal axes can be defined in the transverse plane of the subject as being the front/back and left/right directions of the subject. A vertical axis may then be defined as being perpendicular to the transverse plane. If a measurement in the images is known, for example the distance between two markers, the sparse cloud 560 may be scaled accordingly. Some points may be discarded if they appear to be in error based on a heuristic, such as points that are outside the edges of the sheet or outside a bounding box sized to accommodate the dimensions of a typical subject.

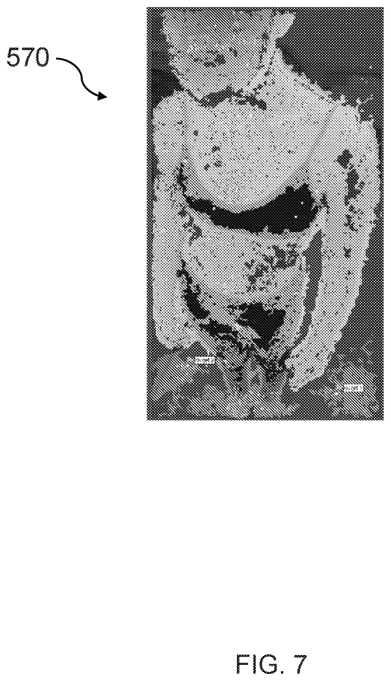

At step 535, the processor 110 and/or the GPU 111 may further refine the 3D model is by generating a depth map of the points, for example using Agisoft Metashape™. The depth map may include distance information about the points, which permits the calculation of additional points that appear in multiple images. FIG. 7 illustrates a depth map 570 of the points, in accordance with one or more non-limiting implementations of the present technology.

At step 540, the processor 110 and/or the GPU 111 may combine the additional points with the previously determined sparse cloud 560 resulting in a dense cloud. The dense cloud may then be further processed by the processor 110 and/or the GPU 111, for example to remove outlier points. The dense cloud may optionally be downsampled, for example by voxelization, depending on the size of the dense cloud.

At step 545, the processor 110 and/or the GPU 111 may further refine the 3D model by generating a triangle mesh based on the point cloud, for example using the Poisson surface reconstruction algorithm. One or more additional processing steps may optionally be performed, such as smoothing to reduce noise, computing the vertex normals of the points, or subdividing the triangles to have more granularity in the data points.

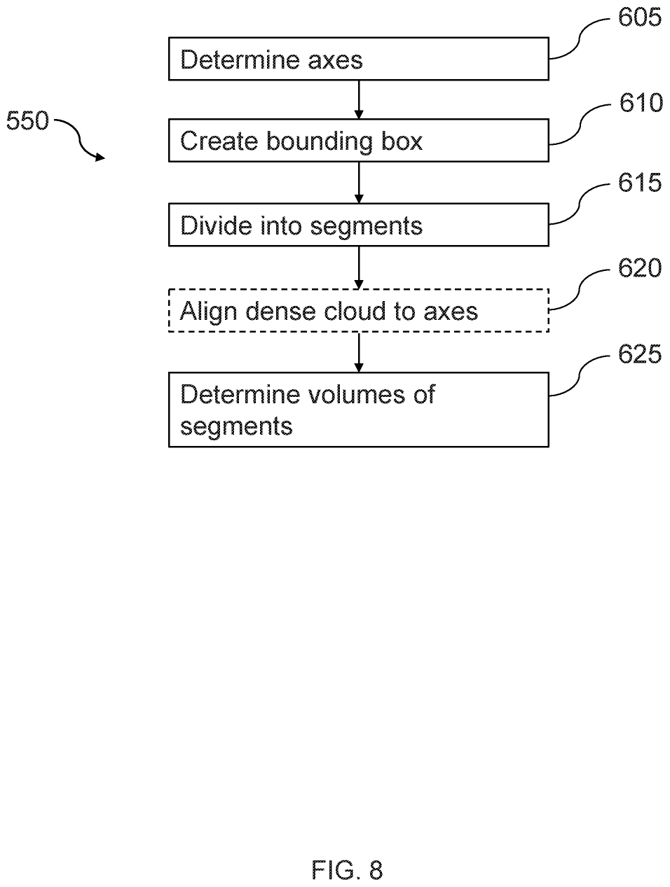

At step 550, the processor 110 and/or the GPU 111 may compute the volumes. An example method 550 of calculating the volumes is shown in detail in FIG. 8.

The process begins at step 605 where the processor 110 and/or the GPU 111 may determine the axes of the subject's body. The axes may be the same axes previously determined at step 530, for example if the subject is facing in a direction that can be determined from the images as described above.

At step 610, the processor 110 and/or the GPU 111 may create a bounding box around the area of interest, which is the torso in the example of a scoliosis subject. In the example of a standing subject, a bounding box of the torso may be approximated by taking 60% of the subject's height as the height of the hips, and the minimum cross section or volume as the height of the neck. The processor 110 and/or the GPU 111 may determine the minimum cross section or volume by determining the first and second derivatives of the cross section or volume with respect to height. Any other suitable method of determining a bounding box of the torso may be used.

At step 615, the processor 110 and/or the GPU 111 may subdivide the segment of the body within the bounding box into segments. For a scoliosis subject, in one example the torso is divided into a number of slices in the transverse plane of the subject, which are further subdivided into quadrants. In one example, 100 slices are used. For the purpose of determining quadrants, the processor 110 and/or the GPU 111 may determine a center point by determining a center of volume of the bottom slice, which corresponds to the height of the hips and defines a central axis of the subject.

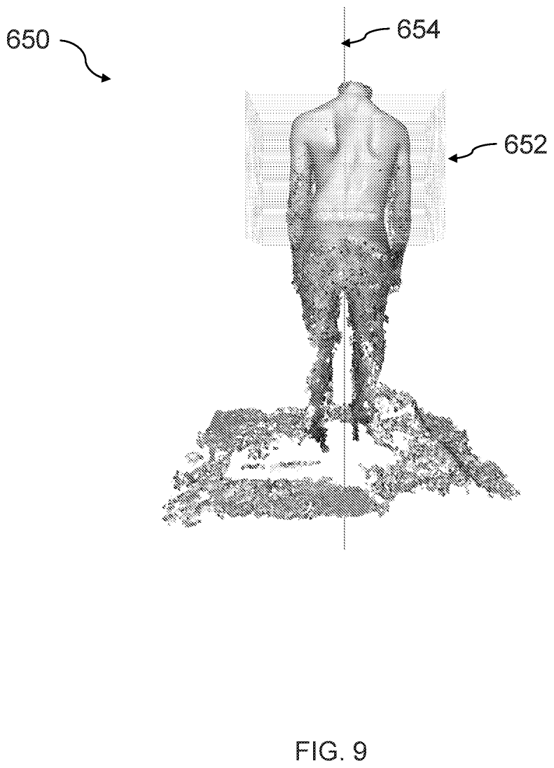

FIG. 9 illustrates the sliced 3D model 650, in accordance with one or more non-limiting implementations of the present technology. As illustrated, the torso is divided into slices 652 and the central axis 654 corresponds to the subject and coincide with the center of volume of the slices 652.

In a scoliosis subject, the hips may be presumed to be symmetrical because they are inferior to (below) the spine, and the spine may be presumed to be asymmetrical, so the center of the body at the hips is a suitable reference.

In some implementations, corrections may be made for hip asymmetries, for example in subjects with unequal leg length or other conditions. Using quadrants defined by axes determined by the direction the subject is facing, and a center determined by the hips, the volumes of the quadrants may permit information about spinal curvature to be determined, as will be discussed below. It should be understood that different shapes of segments may be used, for example when different information is desired or a different part of the body is being diagnosed. In one example, the processor 110 and/or the GPU 111 may divide the slices into front and back halves, or left and right halves, if the information about spinal curvature is only desired in one dimension.

At step 620, the processor 110 and/or the GPU 111 may optionally align the dense cloud to the axes. This can be done, for example, by determining an oriented bounding box and an axis-aligned bounding box for the dense cloud. An oriented bounding box may be the smallest bounding box that may enclose the dense cloud. An axis-aligned bounding box may be the smallest bounding box with edges aligned to the axes that encloses the dense cloud. By comparing the orientations of these two bounding boxes, the orientation of the axes relative to the orientation of the subject may be determined and corrected.

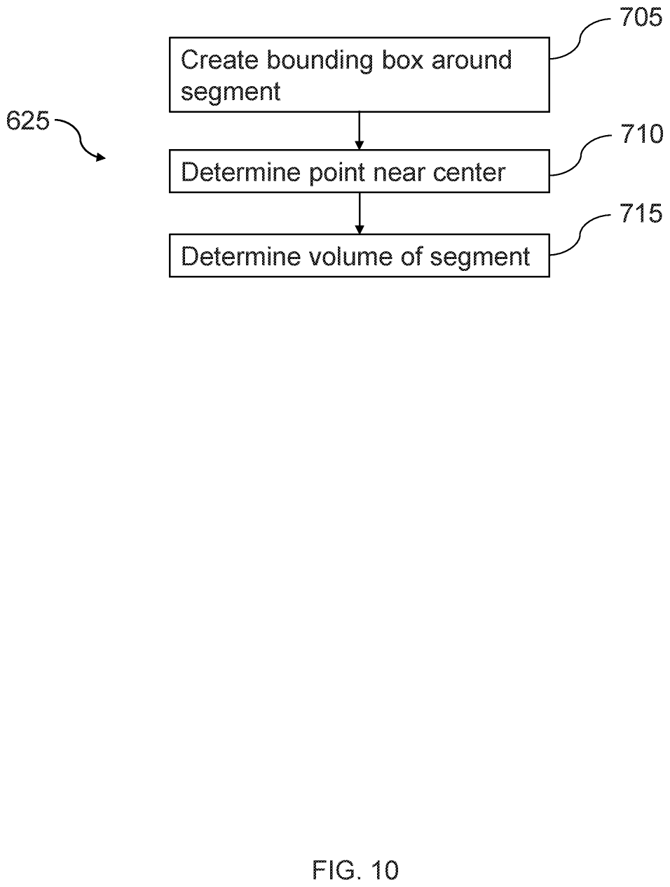

At step 625, the processor 110 and/or the GPU 111 may determine the volumes of the segments. An example method 625 of determining the volume of a segment is shown in greater detail in FIG. 10.

The process commences at step 705 where the processor 110 and/or the GPU 111 may create a bounding box around the segment. In the example of quadrants of transverse slices of the torso, as described above, this may be done based on the definitions of the slices, the axes, and the center that were previously determined.

At step 710, the processor 110 and/or the GPU 111 may determine a point at or near the center of the segment. This may be done, for example, by taking an average of all the points in the segment. Any other suitable method may alternatively be used.

At step 715, the processor 110 and/or the GPU 111 may enlarge the volume from the center point using a flood algorithm, until the volume intersects the points or triangles corresponding to the subject's body or the boundaries of the bounding box. The resulting volume may represent the volume of the segment. It should be understood that, in the case of a thin slice of the image of the subject, the volume may be approximated as a two-dimensional area having the thickness of the slice, and therefore the flood algorithm may instead be used in two dimensions to determine the area of the slice, which may then be used to determine the volume of the segment. It should be understood that any other suitable method may alternatively be used to determine the volume of each segment.

Once the volumes are calculated, the processor 110 and/or the GPU 111 may interpret the volumetric data in a variety of ways. For example, the volumes of adjacent quadrants for each slice may be summed to determine a left-right or front-back volume distribution. The total volume share of the left, right, front, or back half of the body may be determined. Statistical quantities such as the mean, median, or standard deviation in any set of partial volumes may be significant, depending on the purpose for which the data was collected.

Returning to FIG. 3, at step 330, the processor 110 and/or the GPU 111 may determine a degree of spinal curvature of the subject. In one method, the volumetric data, for example the volumes of the individual segments, are input into a convolutional neural network (CNN) or other suitable machine learning (ML) model. The CNN may include two convolutional layers, a flatten layer, and two dense layers. The CNN may rely on the Adam optimizer. The CNN may be trained on data sets including volumetric measurements of subjects who had a known degree of spinal curvature, for example a known Cobb angle, which may have been determined by a conventional clinical examination using X-rays. The CNN may then process the volumetric data of the subject and output the degree of spinal curvature of the subject, for example the Cobb angle of the subject.

In some implementations, the Cobb angle of a subject may be determined without the need for specialized equipment such as X-ray machines. In some implementations, the Cobb angle of a subject may be determined in a manner that is convenient to the subject. In some implementations, the Cobb angle of a subject may be determined without the need for specialized medical training.

It should be expressly understood that not all technical effects mentioned herein need to be enjoyed in each and every implementation of the present technology. For example, implementations of the present technology may be implemented without the user enjoying some of these technical effects, while other non-limiting implementations may be implemented with the user enjoying other technical effects or none at all.

Some of these steps and signal sending-receiving are well known in the art and, as such, have been omitted in certain portions of this description for the sake of simplicity. The signals can be sent-received using optical means (such as a fiber-optic connection), electronic means (such as using wired or wireless connection), and mechanical means (such as pressure-based, temperature based or any other suitable physical parameter based).

Modifications and improvements to the above-described implementations of the present technology may become apparent to those skilled in the art. The foregoing description is intended to be exemplary rather than limiting.

1. A method of determining a degree of spinal curvature, the method being executed by a processor, the method comprising:
for each segment of a plurality of segments of a torso of a subject:
determining at least one volume of the segment; and
determining the degree of spinal curvature of the subject based on the volumes of the respective segments.

2. The method of claim 1, wherein the at least one volume is a volume of a substantially planar segment oriented in a transverse plane of the subject.

3. The method of claim 2, wherein the at least one volume includes four volumes of the segment in respective quadrants of the transverse plane.

4. The method of claim 3, wherein the respective quadrants are defined relative to a center of a segment located inferior to a spine of the subject.

5. The method of claim 4, wherein determining the degree of spinal curvature comprises determining a Cobb angle.

6. The method of claim 5, wherein determining the degree of spinal curvature includes using a convolutional neural network.

7. The method of claim 6, wherein the convolutional neural network includes two convolutional layers, a flatten layer, and two dense layers, using an Adam optimizer.

8. The method of claim 7, further comprising determining the plurality of segments, wherein said determining the plurality of segments comprises:
obtaining a plurality of images of the torso of the subject;
determining a shape of the torso based on the plurality of images; and
dividing the shape of the torso into the plurality of segments.

9. The method of claim 8, wherein at least some of the plurality of images contain at least one reference marker of a plurality of reference markers, and determining the shape of the torso includes determining a position of at least one point on the torso with respect to the plurality of reference markers.

10. The method of claim 9, wherein determining the shape of the torso comprises generating a three-dimensional (3D) model of the torso.

11. A method of determining a degree of spinal curvature, the method being executed by a processor, the method comprising:
receiving a plurality of images of at least a torso of a subject;
determining a plurality of segments of the torso;
determining respective volumes of the plurality of segments of the torso based on the plurality of images;
determining the degree of spinal curvature of the subject based on respective shares of the respective volumes of the segments; and
outputting the determined degree of spinal curvature.

12. The method of claim 11, wherein said determining the degree of spinal curvature comprises using a convolutional neural network.

13. The method of claim 12, wherein the convolutional neural network includes two convolutional layers, a flatten layer, and two dense layers, using an Adam optimizer.

14. The method of claim 13, wherein determining the plurality of segments comprises:
determining a shape of the torso based on the plurality of images; and
dividing the shape of the torso into the plurality of segments.

15. The method of claim 14, wherein at least some of the plurality of images include at least one of a plurality of reference markers, and wherein determining the shape of the torso includes determining a position of at least one point on the torso with respect to the plurality of reference markers.

16. The method of 15, wherein determining the position of the at least one point on the torso includes generating a depth map of a plurality of points on the torso.

17. The method of claim 16, wherein determining the shape of the torso comprises generating a three-dimensional (3D) model of the torso.

18. The method of claim 17, wherein the plurality of segments are substantially planar segments each oriented in a transverse plane of the subject.

19. The method of claim 18, wherein each one of the plurality of segments is located in one quadrant of the transverse plane.

20. The method of claim 19, wherein the quadrant is defined relative to a center of a segment located inferior to a spine of the subject.

21. The method of claim 20, wherein the plurality of images include at least one video recording.

22. A system for determining a degree of spinal curvature, the system comprising:
a processor;
a non-transitory storage medium operatively connected to the processor, the non-transitory storage medium comprising computer-readable instructions;
the processor, upon executing the instructions, being configured to:
for each segment of a plurality of segments of a torso of a subject:
determine at least one volume of the segment; and
determine the degree of spinal curvature of the subject based on the volumes of the respective segments.

23. The system of claim 22, wherein the at least one volume is a volume of a substantially planar segment oriented in a transverse plane of the subject.

24. The system of claim 23, wherein the at least one volume includes four volumes of the segment in respective quadrants of the transverse plane.

25. The system of claim 24, wherein the respective quadrants are defined relative to a center of a segment located inferior to a spine of the subject.

26. The system of claim 25, wherein the processor is further configured to determine the degree of spinal curvature by determining a Cobb angle.

27. The system of claim 26, wherein the processor is further configured to determine the degree of spinal curvature by using a convolutional neural network.

28. The system of claim 27, wherein the convolutional neural network includes two convolutional layers, a flatten layer, and two dense layers, using an Adam optimizer.

29. The system of claim 28, wherein the processor is further configured to determine the plurality of segments by:
obtaining a plurality of images of the torso of the subject;
determining a shape of the torso based on the plurality of images; and
dividing the shape of the torso into the plurality of segments.

30. The system of claim 29, wherein at least some of the plurality of images contain at least one reference marker of a plurality of reference markers, and determining the shape of the torso includes determining a position of at least one point on the torso with respect to the plurality of reference markers.

31. The system of claim 30, wherein the processor is further configured to determine the shape of the torso by generating a three-dimensional (3D) model of the torso.

32. A system for determining a degree of spinal curvature, the system comprising:
a processor;
a non-transitory storage medium operatively connected to the processor, the non-transitory storage medium comprising computer-readable instructions;
the processor, upon executing the instructions, being configured to:
receive a plurality of images of at least a torso of a subject;
determine a plurality of segments of the torso;
determine respective volumes of the plurality of segments of the torso based on the plurality of images;
determine the degree of spinal curvature of the subject based on respective shares of the volumes of the segments; and
output the determined degree of the spinal curvature.

33. The system of claim 32, the processor is configured to determine the degree of spinal curvature using a convolutional neural network.

34. The system of claim 33, wherein the convolutional neural network includes two convolutional layers, a flatten layer, and two dense layers, using an Adam optimizer.

35. The system of claim 34, wherein the processor is configured to determine the plurality of segments by:
determining a shape of the torso based on the plurality of images; and
dividing the shape of the torso into the plurality of segments.

36. The system of claim 35, wherein at least some of the plurality of images include at least one of a plurality of reference markers, and wherein the processor is configured to determine the shape of the torso by determining a position of at least one point on the torso with respect to the plurality of reference markers.

37. The system of 36, wherein the processor is further configured to determine the position of the at least one point on the torso by generating a depth map of a plurality of points on the torso.

38. The system of claim 37, wherein the processor is further configured to determine the shape of the torso by generating a three-dimensional (3D) model of the torso.

39. The system of claim 38, wherein the plurality of segments are substantially planar segments each oriented in a transverse plane of the subject.

40. The system of claim 39, wherein each of the plurality of segments is located in one quadrant of the transverse plane.

41. The system of claim 40, wherein the quadrant is defined relative to a center of a segment located inferior to a spine of the subject.

42. The system of claim 41, wherein the plurality of images include at least one video recording.
